(19) United States
(12) Patent Application Publication
(10) Pub. No.: US 2019/0345196 A1
(43) Pub. Date: Nov. 14, 2019

(54) SYSTEMS AND METHODS FOR QUANTIFYING AND MODIFYING PROTEIN VISCOSITY

(71) Applicant: REGENERON PHARMACEUTICALS, INC., Tarrytown, NY (US)

(72) Inventors: Xiaobin Xu, Tarrytown, NY (US); Aming Zhang, Tarrytown, NY (US); Yuan Cao, Tarrytown, NY (US)

(21) Appl. No.: 16/407,414

(22) Filed: May 9, 2019

Related U.S. Application Data
(60) Provisional application No. 62/669,440, filed on May 10, 2018.

(51) Int. Cl. C07K 1/36; C12N 15/85; C07K 1/22; G01N 33/68; G01N 1/40; C12N 15/01

(52) U.S. Cl. CPC C07K 1/36; C12N 15/85; C07K 1/22; G01N 2560/00; G01N 1/4005; C12N 15/01; G01N 2001/4016; G01N 33/6848

(57) ABSTRACT

Systems and methods for determining regions of proteins that contribute to the viscosity of formulations of those proteins are provided. Methods for modifying the viscosity of concentrated protein formulations are also provided.

### CROSS-REFERENCE TO RELATED APPLICATIONS

This application claims benefit of and priority to U.S. Provisional Patent Application No. 62/669,440 filed on May 10, 2018, which is incorporated by reference in its entirety.

### TECHNICAL FIELD OF THE INVENTION

The invention is generally related to methods for predicting viscosity of high concentration therapeutic antibodies.

### BACKGROUND OF THE INVENTION

Monoclonal antibodies are a rapidly growing class of biological therapeutics. Monoclonal antibodies have a wide range of indications including inflammatory diseases, cancer, and infectious diseases. The number of commercially available monoclonal antibodies is increasing at a rapid rate, with ˜70 monoclonal antibody products predicted to be on the market by 2020 (Ecker, D. M, et al., mAbs, 7:9-14 (2015)).

Currently, the most commonly utilized route of administration of therapeutic antibodies is intravenous (IV) infusion. However, subcutaneous injection is being increasingly used for patients with chronic diseases who require frequent dosing. Ready-to-use pre-filled syringes or auto-injector pens allow patients to self-administer therapeutic antibodies. Antibody formulations for subcutaneous injection are typically more concentrated than IV infusion since subcutaneous injection is one bolus administration (typically 1-1.5 mL) in contrast to a slow infusion of antibody over time in the case of IV infusion.

A common challenge encountered with the production of highly concentrated therapeutic monoclonal antibodies is high viscosity (Tomar, D. S., et al., mAbs, 8:216-228 (2016)). High viscosity can cause increased injection time and increased pain at the site of the injection. In addition to problems with administration, highly viscous antibodies also pose problems during bioprocessing of the antibody solution. High viscosity can increase processing time, destabilize the drug product, and increase manufacturing costs. Short range electrostatic and/or hydrophobic protein-protein interactions and electroviscous effects can influence concentration-dependent viscosity behavior of antibodies.

Characterizing the conformation and structural dynamics of an antibody can be a major analytical challenge. Many available structural techniques are either highly sophisticated, requiring very specialized skills and large amounts of sample (>μM quantities), or are of low resolution, making detailed structural analysis difficult. As a result, it is desirable to have techniques available that can probe protein structure with low sample requirements, good resolution, and relatively fast turnaround time.

Therefore, it is an object of this invention to provide methods for identifying regions of proteins that contribute to the viscosity of formulations of that protein.

It is another object of the invention to provide methods for modifying viscosity of concentrated protein solutions.

### SUMMARY OF THE INVENTION

Systems and methods for determining regions of proteins that contribute to the viscosity of formulations of those proteins are provided. Methods for modifying the viscosity of concentrated protein formulations are also provided.

One embodiment provides a method for identifying regions in a protein that contribute to the viscosity of the protein by microdialysing samples of the protein in a microdialysis cartridge against a buffer containing deuterium for at least two different time periods. The microdialysis is subsequently quenched. The quenched samples are then analyzed using an hydrogen/deuterium exchange mass spectrometry system to determine regions of the protein in the sample that have reduced levels of deuterium relative to other regions of the protein. The regions of the protein that have reduced levels of deuterium contribute to the viscosity of the protein.

In certain embodiments, the samples of protein have a concentration of between 10 mg/mL to 200 mg/mL of the protein.

In some embodiments, the samples of protein are microdialysed in a buffer having a pH between 5.0 and 7.5. A preferred buffer for the samples of protein is 10 mM Histidine at pH 6.0. An exemplary deuterium containing buffer includes deuterium in 10 mM Histidine at pH 6.0. Typically, the microdialysis is performed at 2 to 6° C., preferably at 4° C. In some embodiments the microdialysis is performed at 20 to 25° C. Different samples can be dialysed for different lengths of time, for example one sample can be dialysed for 4 hours and another sample can be microdialysed for 24 hours. In some embodiments, the samples are dialysed for 30 min., 4 hours, 24 hours or overnight, i.e., 26 hours.

In certain embodiments, the quenching step is typically performed at −2 to 2° C. for 1 to 5 minutes.

In some embodiments, the method includes the step of digesting the protein into peptides before mass spectrometry analysis.

Another embodiment provides a method of modifying the viscosity of a protein drug, by identifying regions of the protein drug that contribute to the viscosity of the protein drug according to the disclosed methods and modifying the regions of the protein drug that are identified as contributing to the viscosity of the protein drug to modify the viscosity of the protein drug. The regions identified as contributing to the viscosity of the drug can be modified by substituting one or more amino acids in the at least one region to reduce or increase the viscosity as desired.

The protein or protein drug can be an antibody, a fusion protein, a recombinant protein, or a combination thereof. In one embodiment, the protein drug is a concentrated monoclonal antibody.

### BRIEF DESCRIPTION OF THE DRAWINGS

FIG. 1A is a line graph showing viscosity (cP) of mAb1 as a function of concentration (mg/mL). FIG. 1B is a line graph showing viscosity (cP) of mAb2 as a function of concentration (mg/mL).

FIG. 2A-2F is a schematic of an exemplary microdialysis based HDX-MS protocol. Microdialysis cartridges (FIG. 2A) are obtained, D₂O buffer is added to a deep-well plate (FIG. 2B), samples are loaded into the microdialysis cartridges (FIG. 2C), the microdialysis cartridges are loaded into the deep-well plate (FIG. 2D), samples are incubated in the D₂O buffer for various time points (FIG. 2E), and the samples are removed for MS analysis (FIG. 2F).

FIGS. 3A-3F are exemplary spectrograms of deuterium uptake over time in non-CDR mAb1 samples at 15 mg/mL concentrations (FIGS. 3A-3C) and 120 mg/mL concentrations (FIGS. 3D-3F) 0 hours (FIGS. 3A and 3D), 4 hours (FIGS. 3B and 3E), or 24 hours (FIGS. 3C and 3F) after deuterium incubation. FIGS. 3G-3L are spectrograms of deuterium uptake over time in non-CDR mAb1 samples at 15 mg/mL concentrations (FIGS. 3G-31) and 120 mg/mL concentrations (FIGS. 3J-3L) 0 hours (FIGS. 3G and 3J), 4 hours (FIGS. 3H and 3K), or 24 hours (FIGS. 3I and 3L) after deuterium incubation. FIGS. 3M and 3N are deuterium uptake plots showing deuterium uptake % versus time (hrs) for 15 mg/mL (♦) and 120 mg/mL ( [symbol] ) for mAb1 HC36-47 and mAb1 LC48-53.

FIGS. 4A-4B and 4E-4F are butterfly plots showing relative deuterium uptake in heavy chain CDR regions for mAb1 (FIGS. 4A and 4E) and mAb2 (FIGS. 4B and 4F) after 4 hours or 24 hours of deuterium incubation. The top plots represent 120 mg/mL sample concentration and the bottom plots represent 15 mg/mL sample concentration. The X axis represents peptide number and the Y axis represents differential deuterium uptake (%). FIGS. 4C-4D and 4G-4H are residual plots showing relative deuterium uptake in heavy chain CDR regions for mAb1 (FIGS. 4C and 4G) and mAb2 (FIGS. 4D and 4H) after 4 hours or 24 hours of deuterium incubation. The top plots represent 120 mg/mL sample concentration and the bottom plots represent 15 mg/mL sample concentration. The X axis represents peptide number and the Y axis represents differential deuterium uptake (%). FIGS. 4G-4H are residual plots of deuterium uptake in mAb1 light chain (FIG. 4G) and mAb2 light chain (FIG. 4H) after 4 hours or 24 hours of incubation. The X axis represents peptide number and the Y axis represents differential deuterium uptake (%).

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “protein” refers to a molecule comprising two or more amino acid residues joined to each other by a peptide bond. Protein includes polypeptides and peptides and may also include modifications such as glycosylation, lipid attachment, sulfation, gamma-carboxylation of glutamic acid residues, alkylation, hydroxylation and ADP-ribosylation. Proteins can be of scientific or commercial interest, including protein-based drugs, and proteins include, among other things, enzymes, ligands, receptors, antibodies and chimeric or fusion proteins. Proteins are produced by various types of recombinant cells using well-known cell culture methods, and are generally introduced into the cell by transfection of genetically engineering nucleotide vectors (e.g., such as a sequence encoding a chimeric protein, or a codon-optimized sequence, an intronless sequence, etc.), where the vectors may reside as an episome or be intergrated into the genome of the cell.

“Antibody” refers to an immunoglobulin molecule consisting of four polypeptide chains, two heavy (H) chains and two light (L) chains inter-connected by disulfide bonds. Each heavy chain has a heavy chain variable region (HCVR or VH) and a heavy chain constant region. The heavy chain constant region contains three domains, CH1, CH2 and CH3. Each light chain has a light chain variable region and a light chain constant region. The light chain constant region consists of one domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. The term antibody also includes bispecific antibody, which includes a heterotetrameric immunoglobulin that can bind to more than one different epitope. Bispecific antibodies are generally described in US Patent Application Publication No. 2010/0331527, which is incorporated by reference into this application.

A “CDR” or complementarity determining region is a region of hypervariability interspersed within regions that are more conserved, termed “framework regions” (FR). The FRs may be identical to the human germline sequences, or may be naturally or artificially modified.

As used herein, “viscosity” refers to the rate of transfer of momentum of liquid. It is a quantity expressing the magnitude of internal friction, as measured by the force per unit area resisting a flow in which parallel layers unit distance apart has unit speed relative to one another. In liquids, viscosity refers to the “thickness” of a liquid.

The term “HDX-MS” refers to hydrogen/deuterium exchange mass spectrometry.

As used herein, “dialysis” is a separation technique that facilitates the removal of small, unwanted compounds from macromolecules in solution by selective and passive diffusion through a semi-permeable membrane. A sample and a buffer solution (called the dialysate, usually 200 to 500 times the volume of the sample) are placed on opposite sides of the membrane. Sample molecules that are larger than the membrane-pores are retained on the sample side of the membrane, but small molecules and buffer salts pass freely through the membrane, reducing the concentration of those molecules in the sample. Once the liquid-to-liquid interface (sample on one side of the membrane and dialysate on the other) is initiated, all molecules will try to diffuse in either direction across the membrane to reach equilibrium. Dialysis (diffusion) will stop when equilibrium is achieved. Dialysis systems are also used for buffer exchange.

The term “microdialysis” refers to the dialysis of samples having a volume of less than one milliliter.

“D₂O” is an abbreviation for deuterated water. It is also known as heavy water or deuterium oxide. D₂O contains high amounts of the hydrogen isotope deuterium instead of the common hydrogen isotope that makes up most of the hydrogen in normal water. Deuterium is an isotope of hydrogen that is twice as heavy due to an added neutron.

### II. Methods for Identifying Regions of Proteins that Contribute to Viscosity

The development of highly concentrated therapeutic monoclonal antibodies is paramount for subcutaneous delivery of monoclonal antibody therapeutics. However, high viscosity is a concern in the production of concentrated monoclonal antibody therapeutics. There is a need to develop computational and experimental tools to rapidly and efficiently determine the concentration-dependent viscosity behavior of candidate therapeutics early in the development process.

A. Microdialysis-Hydrogen/Deuterium Exchange Mass Spectrometry

During the course of development, a therapeutic monoclonal antibody can exhibit unusually high viscosity, for example at concentrations>100 mg/mL when compared to other similar monoclonal antibodies. This may be due to the characteristic short range electrostatic and/or hydrophobic protein-protein interactions of the monoclonal antibody under high concentrations. Hydrogen/deuterium exchange mass spectrometry (HDX-MS) is a useful tool to investigate protein conformation, dynamics, and interactions. However, the conventional dilution labeling HDX-MS analysis has a limitation on analyzing unusual behaviors that only occur at high protein concentrations. In order to probe protein-protein interactions governing high viscosity of monoclonal antibodies at a high protein concentration with HDX-MS, a passive, microdialysis based HDX-MS method to achieve HDX labeling without D₂O buffer dilution was developed, which allows for the profiling of characteristic molecular interactions at different protein concentrations. The use of a microdialysis plate significantly reduced the consumption of samples and D₂O compared to the traditional dialysis devices. This method was applied to investigate protein-protein interactions at a high concentration of monoclonal antibodies which have very high viscosity.

Proteins with high viscosity behavior can be optimized to reduce or eliminate the high viscosity behavior. Methods of optimizing protein drugs or antibodies include but are not limited to optimizing the amino acid sequence to reduce viscosity, altering the pH or salt content of the formulation, or adding an excipient.

In one embodiment, multiple therapeutic protein or antibody formulations can be tested to determine the most promising candidate to move forward in production. High and low concentration samples of each protein or antibody are produced. In one embodiment, a high protein or antibody concentration is >50 mg/mL. The high concentration can be 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, or >200 mg/mL. In one embodiment, a low antibody concentration is <15 mg/mL. The low concentration can be 15 mg/mL, 10 mg/mL, 9 mg/mL, 8 mg/mL, 7 mg/mL, 6 mg/mL, 5 mg/mL, 4 mg/mL, 3 mg/mL, 2 mg/mL, 1 mg/mL, 0.5 mg/mL, or <0.5 mg/mL.

More details in the steps of the disclosed methods are provided below.

1. Hydrogen/Deuterium Exchange

Hydrogen/deuterium exchange is a phenomenon in which hydrogen atoms at labile positions in proteins spontaneously change places with hydrogen atoms in the surrounding solvent which contains deuterium ions (Houde, D. and Engel, J. R., Methods Mol Biol, 988:269-289 (2013)). HDX takes advantage of the three types of hydrogens in proteins: those in carbon-hydrogen bonds, those in side-chain groups, and those in amide functional groups (also called backbone hydrogens). The exchange rates of hydrogens in carbon-hydrogen bonds are too slow to observe, and those of side-chain hydrogens (e.g., OH, COOH) are so fast that they back-exchange rapidly when the reaction is quenched in H₂O-based solution, and the exchange is not registered. Only the backbone hydrogens are useful for reporting protein structure and dynamics because their exchange rates are measureable and reflect hydrogen bonding and solvent accessibility. Amide hydrogens play a key role in the formation of secondary and tertiary structure elements. Measurements of their exchange rates can be interpreted in terms of the conformational dynamics of individual higher-order structural elements as well as overall protein dynamics and stability.

Exchange rates reflect on the conformational mobility, hydrogen bonding strength, and solvent accessibility in protein structure. Information about protein conformation and, most importantly, differences in protein conformation between two or more forms of the same protein can be extracted by monitoring the exchange reaction. The exchange rate is temperature dependent, decreasing by approximately a factor of ten as the temperature is reduced from 25° C. to 0° C. Consequently, under pH 2-3 and at 0° C. (commonly referred to as “quench conditions”) the half-life for amide hydrogen isotopic exchange in an unstructured polypeptide is 30-90 min, depending on the solvent shielding effect caused by the side chains. Hydrogen has a mass of 1.008 Da and deuterium (the second isotope of hydrogen) has a mass of 2.014 Da, hydrogen exchange can be followed by measuring the mass of a protein with a mass spectrometer.

In one embodiment, hydrogen/deuterium exchange rate is used to determine viscosity behavior of protein or antibody therapeutics.

2. Microdialysis

Classical continuous HDX labeling via dilution is not applicable in the analysis of highly concentrated protein solutions. One embodiment herein provides an alternative method of HDX labeling for the use with high concentration protein solutions. HDX labeling in a microdialysis plate facilitates the analysis of highly concentrated protein solutions. In addition, the use of a microdialysis plate reduces the consumption of samples and D₂O compared to traditional dialysis devices (Houde, D., et al., J Am Soc Mass Spectrom, 27(4):669-76 (2016)). The microdialysis plate can be a commercially available microdialysis plate, for example Pierce™ 96-well Microdialysis Plate.

In one embodiment, microdialysis HDX exchange is used to analyze highly concentrated protein solutions. The samples are loaded into the microdialysis cartridge of the microdialysis plate. D₂O buffer is added to a deep-well plate or other suitable vessel. The microdialysis cartridges containing the protein samples are added to the buffer and allowed to incubate for at least 4 hours. The samples can incubate for 0.5, 1, 2, 3, 4, 5, 6, 7, 8, 9, 10, 11, 12, 13, 14, 15, 16, 17, 18, 19, 20, 21, 22, 23, 24, or more than 24 hours. The dialysis system allows for passive diffusion of the buffer into the cartridge containing the sample so as to not dilute out the sample as is common in traditional continuous HDX labeling wherein large quantities of buffer are required. During the incubation step, deuterium in the D₂O buffer enters into the cartridge containing the sample and is exchanged with hydrogens in the backbone amides of the protein samples. After the incubation step, samples are collected from the microdialysis cartridge.

3. Sample Preparation

Once the dialyzed samples are removed from the microdialysis cartridge, the HDX reaction can be terminated by quenching the samples. In one embodiment, quenching is achieved by adding quench buffer to the samples. The quenching buffer can contain 6M GlnHCl and 0.6M TCEP in H₂O, pH 2.5. In one embodiment, the quenching buffer contains 8 M Urea, 0.6M TCEP in H₂O, pH 2.5. In another embodiment, the pH of the final quenched solution is 2.5.

In one embodiment, decreasing the reaction temperature can also quench the HDX reaction. The reaction can be carried out at 0° C. The exchange rate decreases by a factor of ten as the temperature is reduced from 25° C. to 0° C. In one embodiment, the quenching reaction is carried out at or below 0\hat{∘}° C.

After quenching, the samples can be diluted for downstream mass spec analysis. Samples can be diluted in 0.1% formic acid (FA) in H₂O or any other suitable diluent for use in mass spectrometry. The samples are then processed by a mass spectrometer.

4. Mass Spectrometry

Mass spectrometry is used for determining the mass shifts induced by the exchange of hydrogen by deuterium (or vice versa) over time. Hydrogen has a mass of 1.008 Da and deuterium has a mass of 2.014 Da, therefore hydrogen exchange can be followed by measuring the mass of a protein with a mass spectrometer. Proteins or antibodies that have incorporated deuterium will have an increased mass compared to the native protein or antibody that has not been incubated in D₂O. Generally, the level of exchanged hydrogen reflects the flexibility, solvent accessibility, and hydrogen bonding interactions in protein structures.

In some embodiments on-line digestion is employed to cleave larger proteins or antibodies into smaller fragments or peptides. Commonly used enzymes for on-line digestion include but are not limited to pepsin, trypsin, trypsin/Lys-C, rLys-C, Lys-C, and Asp-N.

In one embodiment, the digested proteins or antibodies are subjected to mass spectrometry analysis. Methods of performing mass spectrometry are known in the art. See for example (Aeberssold, M., and Mann, M., Nature, 422:198-207 (2003)) Commonly utilized types of mass spectrometry include but are not limited to tandem mass spectrometry (MS/MS), electrospray ionization mass spectrometry, liquid chromatography-mass spectrometry (LC-MS), and Matrix-assisted laser desorption/ionization (MALDI).

### III. Methods for Modifying Protein Viscosity

One embodiment provides a method of modifying the viscosity of a protein drug, by identifying regions of the protein drug that contribute to the viscosity of the protein drug according to the disclosed methods and modifying the regions of the protein drug that are identified as contributing to the viscosity of the protein drug to modify the viscosity of the protein drug. The regions identified as contributing to the viscosity of the drug can be modified by substituting one or more amino acids in the at least one region to reduce or increase the viscosity as desired.

For example, the light chain, heavy chain, or complementarity determining regions of an antibody can be modified to reduce the viscosity of concentrated formulations of the antibody. An exemplary concentrated formulation has a concentration of antibody that is greater than 50 mg/mL, preferably 100 mg/mL or greater.

Other modifications of the protein or antibody drug include chemical modifications to amino acids in the region of the protein or antibody determined to contribute to the viscosity of the protein or antibody drug.

In one embodiment the protein, antibody, or drug product is or contains one or more proteins of interest suitable for expression in prokaryotic or eukaryotic cells. For example, the protein of interest includes, but is not limited to, an antibody or antigen-binding fragment thereof, a chimeric antibody or antigen-binding fragment thereof, an ScFv or fragment thereof, an Fc-fusion protein or fragment thereof, a growth factor or a fragment thereof, a cytokine or a fragment thereof, or an extracellular domain of a cell surface receptor or a fragment thereof. Proteins of interest may be simple polypeptides consisting of a single subunit, or complex multisubunit proteins comprising two or more subunits. The protein of interest may be a biopharmaceutical product, food additive or preservative, or any protein product subject to purification and quality standards.

In some embodiments, the protein of interest is an antibody, a human antibody, a humanized antibody, a chimeric antibody, a monoclonal antibody, a multispecific antibody, a bispecific antibody, an antigen binding antibody fragment, a single chain antibody, a diabody, triabody or tetrabody, a dual-specific, tetravalent immunoglobulin G-like molecule, termed dual variable domain immunoglobulin (DVD-IG), an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In another embodiment, the antibody comprises a chimeric hinge. In still other embodiments, the antibody comprises a chimeric Fc. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody.

In some embodiments, the antibody is selected from the group consisting of an anti-Programmed Cell Death 1 antibody (e.g. an anti-PD1 antibody as described in U.S. Pat. Appln. Pub. No. US2015/0203579A1), an anti-Programmed Cell Death Ligand-1 (e.g., an anti-PD-L1 antibody as described in U.S. Pat. Appln. Pub. No. US2015/0203580A1), an anti-Dll4 antibody, an anti-Angiopoetin-2 antibody (e.g., an anti-ANG2 antibody as described in U.S. Pat. No. 9,402,898), an anti-Angiopoetin-Like 3 antibody (e.g., an anti-AngPtl3 antibody as described in U.S. Pat. No. 9,018,356), an anti-platelet derived growth factor receptor antibody (e.g., an anti-PDGFR antibody as described in U.S. Pat. No. 9,265,827), an anti-Erb3 antibody, an anti-Prolactin Receptor antibody (e.g., anti-PRLR antibody as described in U.S. Pat. No. 9,302,015), an anti-Complement 5 antibody (e.g., an anti-C5 antibody as described in U.S. Pat. Appln. Pub. No US2015/0313194A1), an anti-TNF antibody, an anti-epidermal growth factor receptor antibody (e.g., an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (e.g., an anti-PCSK9 antibody as described in U.S. Pat. No. 8,062,640 or 9,540,449), an Anti-Growth and Differentiation Factor-8 antibody (e.g. an anti-GDF8 antibody, also known as anti-myostatin antibody, as described in U.S. Pat No. 8,871,209 or 9,260,515), an anti-Glucagon Receptor (e.g. anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat No. 8,735,095 or 8,945,559), an anti-interleukin 6 receptor antibody (e.g., an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g., anti-IL33 antibody as described in U.S. Pat. No. 9,453,072 or 9,637,535), an anti-Respiratory syncytial virus antibody (e.g., anti-RSV antibody as described in U.S. Pat. No. 9,447,173), an anti-Cluster of differentiation 3 (e.g., an anti-CD3 antibody, as described in U.S. Pat. Nos. 9,447,173 and 9,447,173, and in U.S. Application No. 62/222,605), an anti-Cluster of differentiation 20 (e.g., an anti-CD20 antibody as described in U.S. Pat. No. 9,657,102 and US20150266966A1, and in U.S. Pat. No. 7,879,984), an anti-CD19 antibody, an anti-CD28 antibody, an anti-Cluster of Differentiation-48 (e.g. anti-CD48 antibody as described in U.S. Pat. No. 9,228,014), an anti-Fel d1 antibody (e.g. as described in U.S. Pat. No. 9,079,948), an anti-Middle East Respiratory Syndrome virus (e.g. an anti-MERS antibody as described in U.S. Pat. Appln. Pub. No. US2015/0337029A1), an anti-Ebola virus antibody (e.g. as described in U.S. Pat. Appln. Pub. No. US2016/0215040), an anti-Zika virus antibody, an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. Appln. Pub. No. US2016/0017029 and U.S. Pat. Nos. 8,309,088 and 9,353,176) and an anti-Protein Y antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3×anti-Mucin 16 bispecific antibody (e.g., an anti-CD3×anti-Muc16 bispecific antibody), and an anti-CD3×anti-Prostate-specific membrane antigen bispecific antibody (e.g., an anti-CD3×anti-PSMA bispecific antibody). In some embodiments, the protein of interest is selected from the group consisting of abciximab, adalimumab, adalimumab-atto, ado-trastuzumab, alemtuzumab, alirocumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cemiplimab, cetuximab, denosumab, dinutuximab, dupilumab, durvalumab, eculizumnab, elotuzumab, emicizumab-kxwh, emtansinealirocumab, evinacumab, evolocumab, fasinumab, golimumab, guselkumab, ibritumomab tiuxetan, idarucizumab, infliximab, infliximab-abda, infliximab-dyyb, ipilimumab, ixekizumab, mepolizumab, necitumumab, nesvacumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, sarilumab, secukinumab, siltuximab, tocilizumab, tocilizumab, trastuzumab, trevogrumab, ustekinumab, and vedolizumab.

In some embodiments, the protein of interest is a recombinant protein that contains an Fc moiety and another domain, (e.g., an Fc-fusion protein). In some embodiments, an Fc-fusion protein is a receptor Fc-fusion protein, which contains one or more extracellular domain(s) of a receptor coupled to an Fc moiety. In some embodiments, the Fc moiety comprises a hinge region followed by a CH2 and CH3 domain of an IgG. In some embodiments, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-IRAcP ligand binding region fused to the 11-IR1 extracellular region fused to Fc of hIgG1; see U.S. Pat. No. 6,927,004, which is herein incorporated by reference in its entirety), or a VEGF trap (e.g., aflibercept or ziv-aflibercept, which comprises the Ig domain 2 of the VEGF receptor Flt1 fused to the Ig domain 3 of the VEGF receptor Flk1 fused to Fc of hIgG1; see U.S. Pat. Nos. 7,087,411 and 7,279,159). In other embodiments, an Fc-fusion protein is a ScFv-Fc-fusion protein, which contains one or more of one or more antigen-binding domain(s), such as a variable heavy chain fragment and a variable light chain fragment, of an antibody coupled to an Fc moiety.

In one embodiment, the protein drug is a concentrated monoclonal antibody.

### EXAMPLES

### Example 1. Microdialysis HDX Mass Spectrometry

Materials and Methods

mAb1 and mAb2 were diluted in 10 mM histidine (pH 6.0) to create high concentration samples (120 mg/mL) and low concentration samples (15 mg/mL). 160 μl of each sample was loaded into a microdialysis cartridge. The cartridge was inserted into a deep-well plate containing D₂O buffer and incubated for 4 or 24 hours at 4° C. After incubation, 5 μl of each dialyzed sample was quenched by adding quench buffer to the sample, according to Table 1. Quench buffer contains 6M GlnHCl/0.6 M TCEP in 100% D₂O. The quenching reaction was carried out at 0° C. for 3 minutes. 10 μl of each quenched sample was diluted with 0.1% FA in D₂O, according to Table 1. 70 μl of each sample was loaded onto HDX system.

TABLE 1
Sample buffers and dilution volumes.

| | | Volume of | Volume of | Injection |
|---|---|---|---|---|
| Sample | | Quench Buffer | Dilution Buffer | Amount |
| 120 | mg/mL | 5 μL → 295 μL (2 mg/mL) | 10 μL → 130 μL (0.1 mg/mL) | 70 μL (7 μg) |
| 15 | mg/mL | 5 μL → 70 μL (1 mg/mL) | 20 μL → 120 μL (0.1 mg/mL) | 70 μL (7 μg) |

Results

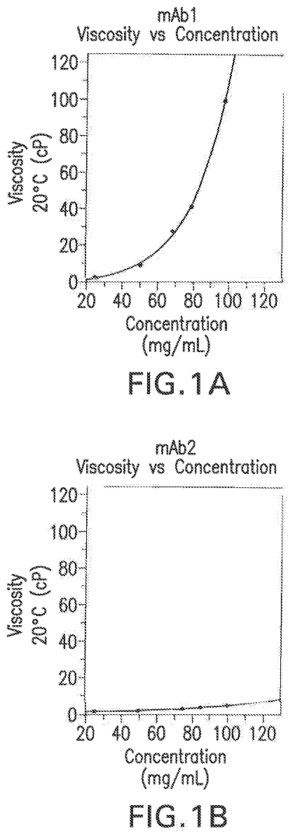

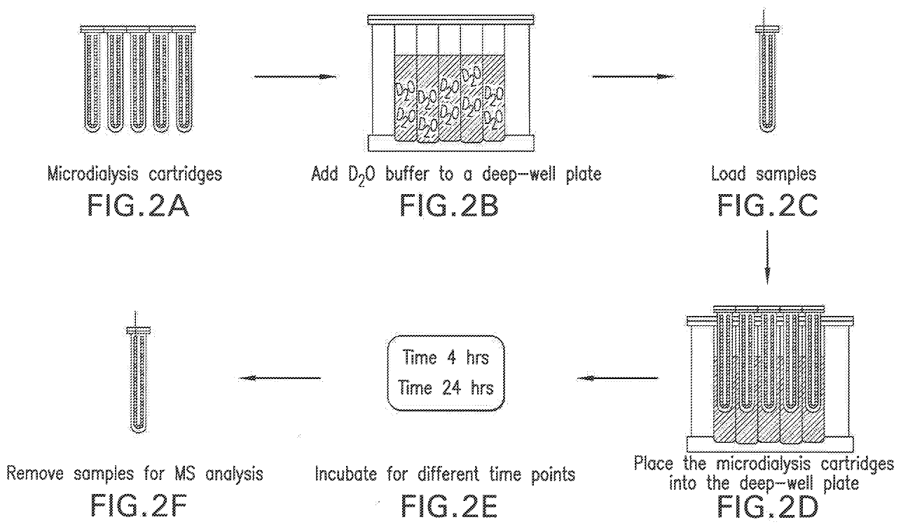

Monoclonal antibody 1 (mAb1) exhibited unusually high viscosity at concentrations>100 mg/mL, when compared to other monoclonal antibodies at the development stage (FIGS. 1A-1B). To probe protein-protein interactions governing the high viscosity of mAb1 at a high protein concentration, a passive, microdialysis based HDX-MS method was developed to achieve HDX labeling without D₂O buffer dilution, which allows profiling molecular interactions at different protein concentrations (FIG. 2A-2F).

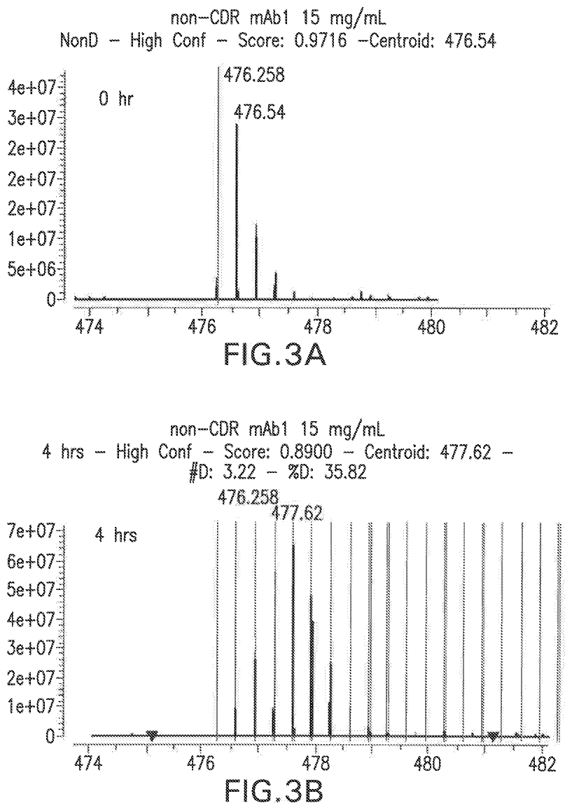

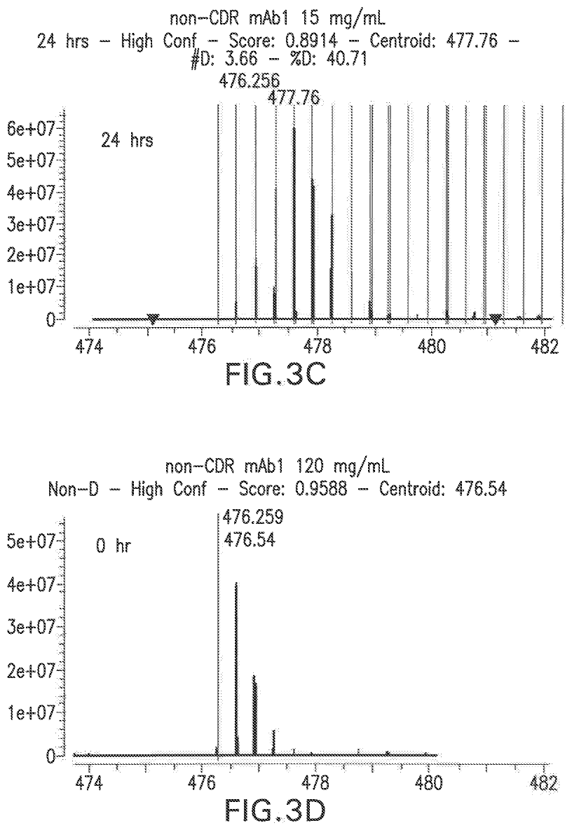

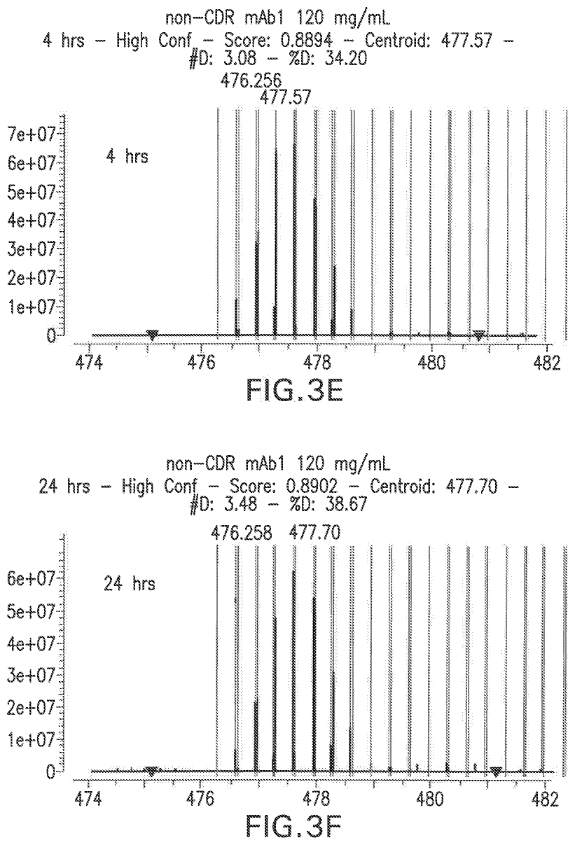

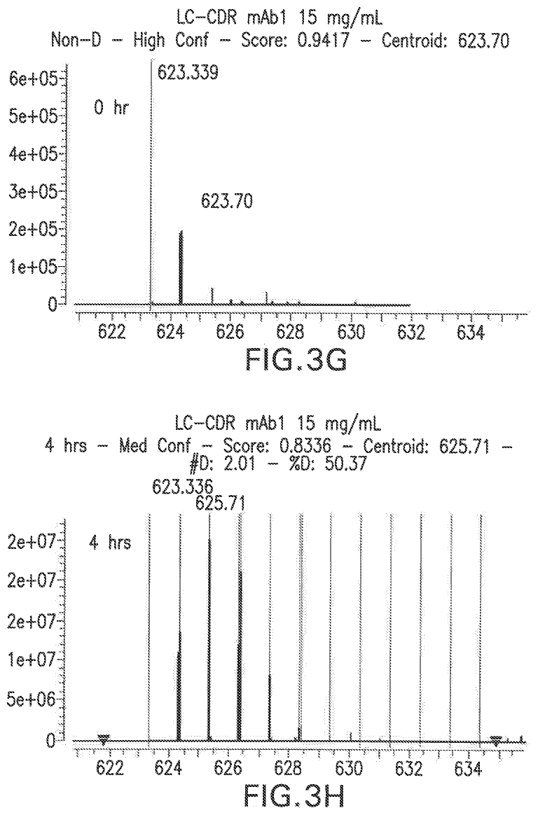

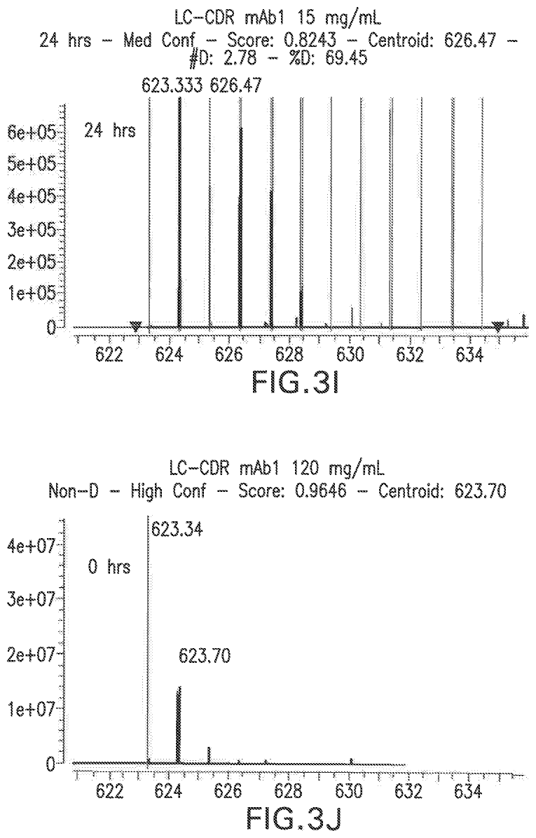

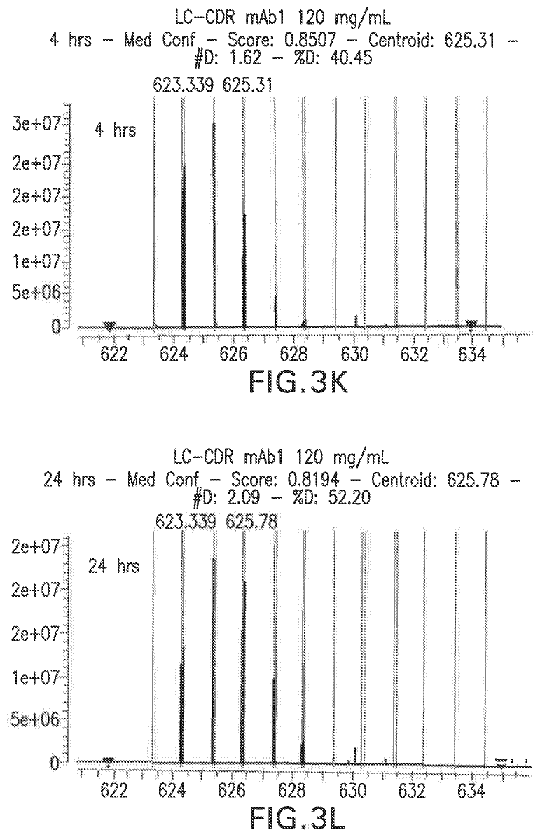

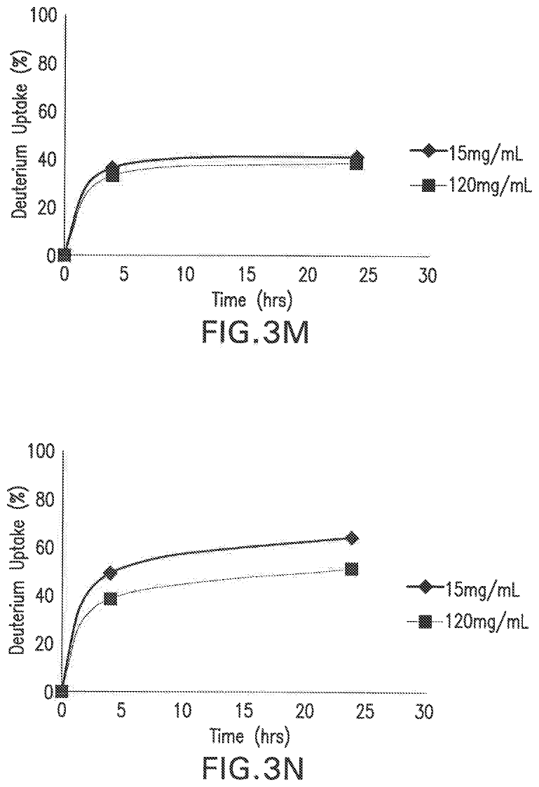

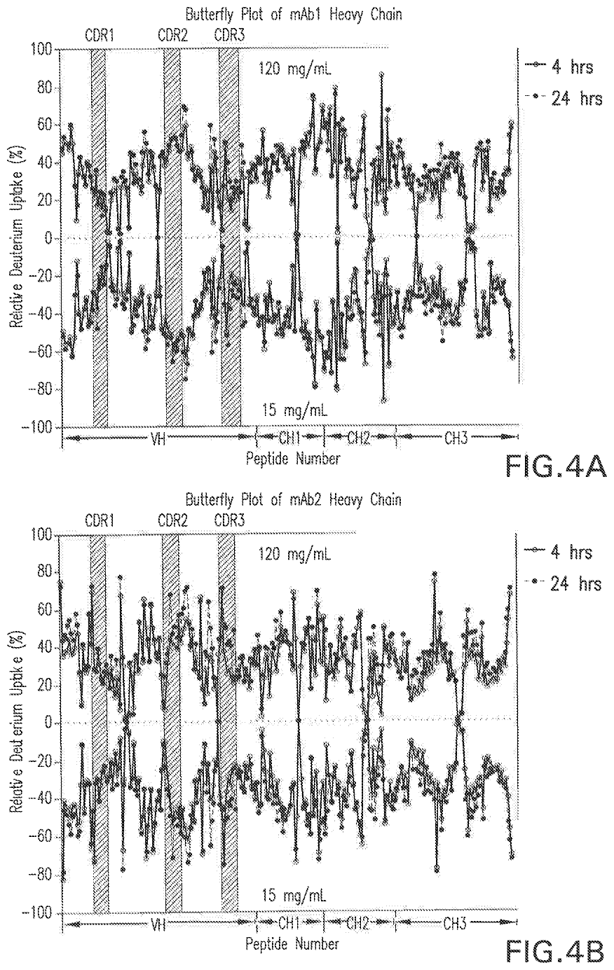

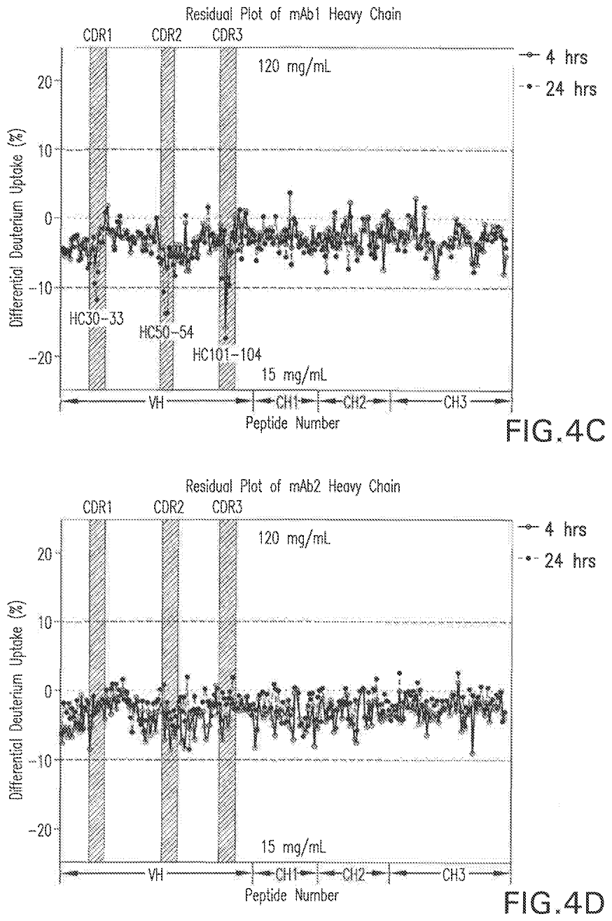

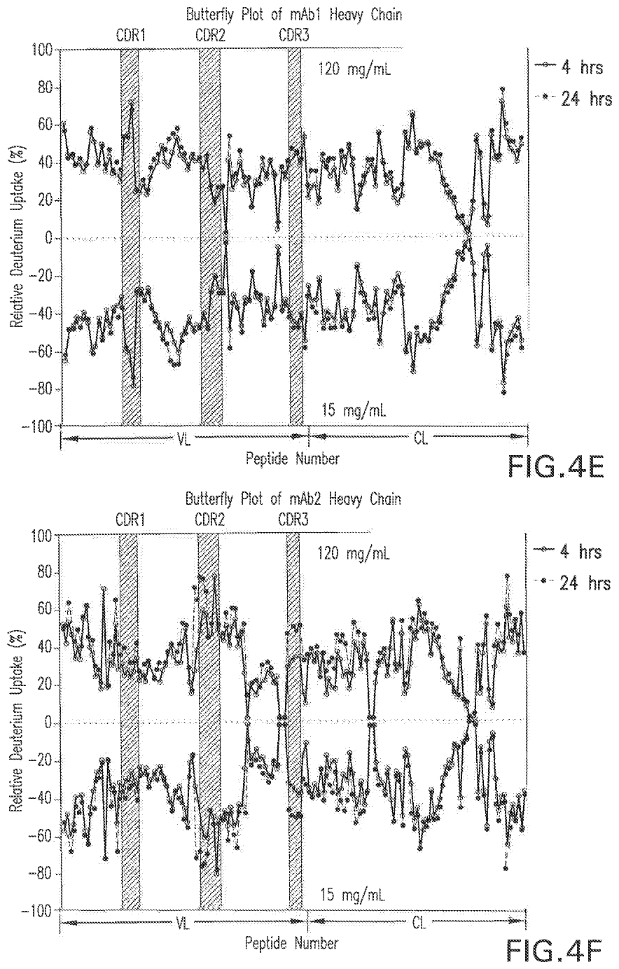

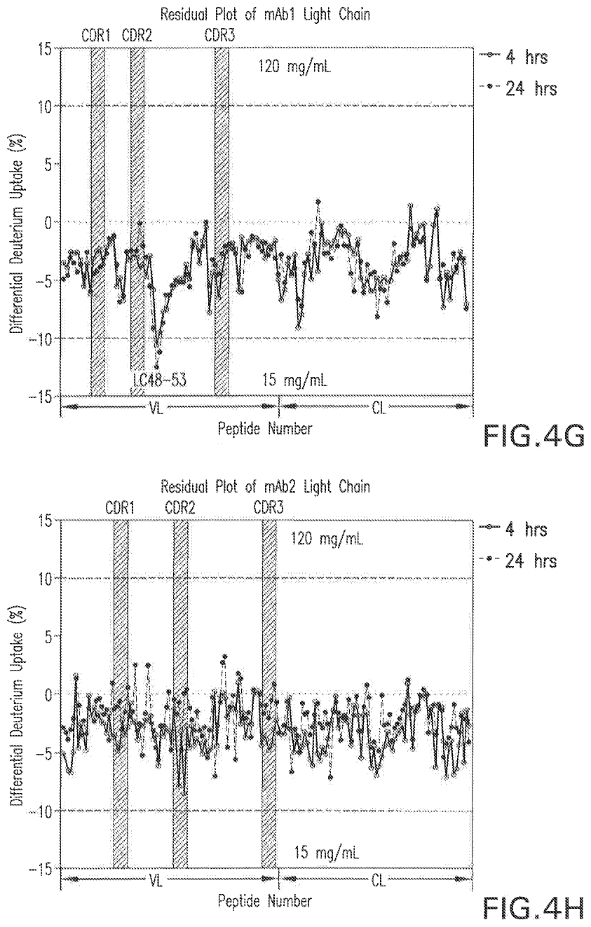

A significant decrease in deuterium was observed in the high concentration samples (120 mg/mL) compared to the control samples (5 mg/mL) at the three heavy chain complementary determining regions and light chain CDR2 for mAb1 (FIGS. 3A-3N, Table 2 and Table 3). This result indicates that these CDRs may be involved in specific intermolecular interactions that could cause the unusually high viscosity observed with mAb1. To confirm that these CDRs are the cause of high viscosity, the disclosed method was applied to investigate protein-protein interactions at high concentration of mAb2 which has the same amino acid sequence as mAb1 except for CDRs and has a low viscosity (FIGS. 4B, 4D, 4F, and 4H). Unlike mAb1, no differential deuterium uptake was observed between the high concentration of mAb2 samples and the low concentration mAb2 samples, further confirming that the CDRs of mAb1 caused the high viscosity at high concentrations.

TABLE 2
Relative deuterium uptake in non-CDR
mAb1 peptide over time.

| | mAb1 non-CDR | |
|---|---|---|
| | Relative Deuterium | |
| | Uptake (%) | |
| Time point | 15 mg/mL | 120 mg/mL/ |
| 0 hr | 0.0% | 0.0% |
| 4 hrs | 36.7% | 33.2% |
| 24 hrs | 41.7% | 38.6% |

TABLE 3
Relative deuterium uptake in LC-
CDR mAb1 peptide over time.

| | mAbl LC-CDR | |
|---|---|---|
| | Relative Deuterium | |
| Time | Uptake (%) | |
| point | 15 mg/mL | 120 mg/mL |
| 0 hr | 0.0% | 0.0% |
| 4 hrs | 49.8% | 39.1% |
| 24 hrs | 65.6% | 52.2% |

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for identifying regions in a protein that contribute to the viscosity of the protein, comprising:
microdialysing samples of the protein in a microdialysis cartridge against a buffer comprising deuterium for at least two different time periods;
subsequently quenching the microdialysis of the samples;
analyzing the quenched samples in an hydrogen/deuterium exchange mass spectrometry system to determine regions of the protein in the sample that have reduced levels of deuterium relative to other regions of the protein, wherein regions of the protein that have reduced levels of deuterium contribute to the viscosity of the protein.

2. The method of claim 1, wherein samples of protein comprise between 10 mg/mL to 200 mg/mL of protein.

3. The method of claim 1, wherein samples of protein in the microdialysing step are in a buffer having a pH between 5.0 and 7.5.

4. The method of claim 1, wherein the samples of protein in the microdialysing step are in 10 mM Histidine at pH 6.0.

5. The method of claim 1, wherein the buffer comprising deuterium comprises 10 mM Histidine at pH 6.0.

6. The method of claim 1, wherein the microdialysis is performed at 2 to 6° C.

7. The method of claim 1, wherein at least one sample is microdialysed for 4 hours and at least another sample is microdialysed for 24 hours.

8. The method of claim 1, wherein the quenching step is performed at −2 to 2° C. for 1 to 5 minutes.

9. The method of claim 1, further comprising digesting the protein into peptides before mass spectrometry analysis.

10. A method of modifying the viscosity of a protein drug, comprising:
identifying regions of the protein drug that contribute to the viscosity of the protein drug according to the method of any one of claim 1,
modifying one or more of the regions identified as contributing to the viscosity of the protein drug to modify the viscosity of the protein drug.

11. The method of claim 10, wherein at least one of the regions identified as contributing to the viscosity of the drug are modified by substituting one or more amino acids in the at least one region.

12. The method of claim 10, wherein the one or more regions identified as contributing to the viscosity of the protein drug are modified to reduce the viscosity of the protein drug.

13. The method of claim 1, wherein the protein is selected from the group consisting of an antibody, a fusion protein, a recombinant protein, or a combination thereof.

14. The method of claim 10, wherein the protein drug is a concentrated monoclonal antibody.

15. The protein drug produced by the method of claim 10.
